(19) United States
(12) Patent Application Publication
(10) Pub. No.: US 2010/0120112 A1
(43) Pub. Date: May 13, 2010

(54) Process of Improved Semi-Static Composting for the Production of a Humectant Substrate of Low Density of Use Thereof in Nurseries and Greenhouses

(75) Inventors: Sergio Ruben Trejo Estrada, Pue. (MX); Julieta Salome Veloz Rendon, Pue. (MX); Minerva Rosas Morales, Pue. (MX); Ana Itzel Reyes Mendez, Pue. (MX)

Correspondence Address: Patent Docket Department;Armstrong Teasdale LLP, One Metropolitan Square, Suite 2600, St. Louis, MO, 63102-2740, US

(73) Assignees: CENTRAL MOTZORONGO S.A. DE C.V., Delegacion Benito Juarez (MX); INSTITUTO POLITECNICO NACIONAL, Delegacion Gustavo A. Madero (MX)

(21) Appl. No.: 12/597,413

(22) PCT Filed: Apr. 23, 2008
(86) PCT No.: PCT/MX2008/000056

§ 371 Date: Jan. 22, 2010

(30) Foreign Application Priority Data
Apr. 26, 2007 (MX) MX/A/2007/005076

(51) Int. Cl. C12P 1/02

(52) U.S. Cl. 435/171

(57) ABSTRACT

This invention includes the improved composting of cane sugar press mud and lignocellulosic materials. The result is a low density humectant substrate (LDHS) for the application in agriculture. The process is developed in homogenized and aerated mechanically semi-static biopiles. The lignocellulose materials are added in a fed batch system, in stages and doses which depend on the type of lignocellulosic material and the quality of required final substrate.

Another objective is to provide, in 8 weeks, a material without pathogenic microorganisms nor weeds, with low density (<0.4 g/ml), high porosity (110%), and high water retention (>90%), applicable as a substrate in horticulture and forestal production in nurseries and greenhouses; or as an humectant and soil-improving agent in agricultural ground and eroded soils. Said substrate has better physical, chemical and biological characteristics for vegetable nutrition than equivalent substrates, such as turf and coconut fibers.

### TECHNICAL FIELD OF THE INVENTION

The present invention in general refers to the area of agricultural biotechnology, namely to the process of agro-industrial residues, and in particular, to a process of improved composting for the production of a low-density humectant substrate for its use in an intensive agricultural production in greenhouses, nurseries and agricultural ground.

### BACKGROUND

The improvement of soils is an urgent need. The well-being of the humanity depends, to a large extent, on its good maintenance. (Wallace, 1998). At present, the value of soil is recognized, as the humanity is able to produce foods thereby. But there is another important reason to participate in the maintenance of soil which is that acts as an environmental filter in order to clean the air and water (Rechcigl¹, 1995).

Actually, soil is not a renewable resource at short term and care must be taken in order to attain future generations may obtain the same benefits of the Earth. (Wallace, 1998). A form to benefit the soil is by the use of composting which is defined as the biological decomposition and stabilization of organic substrates under conditions that allow the development of high temperatures as a result of the biologically produced heat, with the purpose to obtain a stable final product, free of pathogens and weeds (Bertrán, 2004). In general terms, composting is the practice to use organic wastes that through biological reduction, become to an humus or similar substances (Wallace, 1998). The humus is the dark color organic material of soil and has physical and chemical properties which are not subject to a fast decomposition as the residues of plants (Kohnke, 1995). It is a colloidal substance (like glue) containing almost 50% of carbon, 5% of nitrogen and 0.5% of phosphorus, it is chemically combination of modified lignin (more resistant to degradation component of the cell wall of the plants), amino acids (components of proteins) and other nitrogenous components (Kohnke, 1995).

Composting offers several benefits over the simple addition of organic material to soil (Plaster, 2003):
It reduces the weight and volume of organic material making it easier to handle and transport (Plaster, 2003).
It can mitigate the weathering and agronomic restrictions of soils, improving the levels of water retention, nutritional content of the mixture of soil, diminishing the apparent density, and increasing the stability of aggregates of soil (Tilston, 2005).
It reduces the amounts of carbon and nitrogen of materials that are to adhere to soil (Plaster, 2003).
It inhibits microbial pathogens that can be in general or specific form, in the general form the exclusion is through the competition for space. And in the specific form there is the production of inhibiting or toxic metabolites of certain microorganisms.
The application of non-composted wastes can generate the immobilization of nutrients of the plants and produce phytotoxicity (Goyal, 2005).

Several studies of the process of composting have been intensively performed in the last decades, due to the increase of industrial activity and the human population. The most part of the studies have been focused in the use of municipal and industrial wastes for the production of composts.

There are common objectives in the studies on composting: the control of the process of composting, the characterization of composts and criteria for the stabilization of composts. Some strategies have been proposed in order to achieve these objectives. (Bodin, 2005):
To adjust the parameters of the process, composition of the starting mixture of organic material, temperatures during the process, aeration, and frequency of composting, among others.
To alter the initial conditions by the change of the composition or type of material used as sources of carbon and nitrogen.
To modify the process of composting applying nutritious materials of the type of fertilizers in two different times, at the beginning and in the middle of the process, in order to have a mature compost with suitable nitrogen levels.

Strictly speaking, any organic material which may adhere, must be considered as useful harvests for composting (Haug, 1993).

Even so, in the practice the terms enhancers or conditioners and bulk agents have been adopted to certain types of substrates added to composting:
The enhancer or conditioner is an organic or inorganic material which is added with the purpose to improve the pores in the matrix of the compost and/or to increase the biodegradable components (Haug, 1993).
The bulk agent is an organic or inorganic material of enough size to provide a structural support and maintain the air spaces through the matrix of the compost (Haug 1993).

The selection and/or preparation of the raw material and the components of the starting mixture for the compost establish the system of composting to be used. That is why the components of mixture must be adjusted to proportions may allow to have optimal porosity, humidity and nutrients during the process (Goyal, 2005).

### Stages or Phases of the Process of Composting

The system of composting is often divided in three phases, the first and second stages are of high activity and the third is a stabilization or maturation phase. In the first and second phases the method of static, semi-static, shaken windrows or piles can be used, or they can be performed in reactors; since they are characterized by a high demand of oxygen, moderate to high temperatures, the pH is acid by the production of organic acids, a quick reduction of volatile biodegradable solids and disagreeable odors (Haug, 1993).

In the third stage, semi-static or fully static windrows or piles and even closed reactors can be used too. This phase characterized by low temperatures, reduction of the demand of oxygen, and low production of odors. The degradation of materials with low availability is also carried out, there is a decrease in the microbial activity by the effects imposed by a kinetic restriction, the restabilization of low temperatures, the pH is increased by the decomposition of proteins that release ammonium and the final product has a neutral to alkaline pH.

Finally, a mature compost is obtained, wherein phytotoxic compounds have been metabolized and pathogens for the plants have been eliminated. The quality of the generated product depends on the characteristics of the starting material, the design of parameters in the first and second phases and the operative conditions maintained in the system (Haug, 1993).

The grade of maturation of a compost is directed by the intented use or by the final product. Some criteria have been developed in order to measure the grade of stabilization (Rechcigl, 1995):
Decrease of the temperature at the end of the process of composting.
Darkening of the final product.
Analysis of the content of the C:N, 30:1. suitable ratio
Demand of oxygen of the final product in an 1/30 proportion of substrate.
Presence of nitrate with nearly absence of ammonium and starch.
There should not be attraction of insects, nor presence of larvae.
There should not be presence of bad odors which are characteristic in the final product.
   As above described, many parameters have been intended in order to evaluate the stability of compost. Even so, there is not a single method that can be universally applied to all types of compost due to the variability of materials and the process of composting. It has been suggested to use a germination assay to assure the stabilization of compost; when the germination index is greater than 80%, the compost is considered mature and practically free of phytotoxic substances (Rechcigl, 1995).

### Biota of Compost and its Activities

Most of the composts have a microbial load that comes from the ambiance. The representatives of these biological communities are bacteria, actinomycetes and fungi which are normally present when the first stage of composting begins. In the first stage of composting the mesofauna is important too, like worms, millipedes, centipedes, acaruses, beatles, earthworms and thysanurans which break the organic material into small pieces; this preparatory step accelerates the grade of decomposition by the increase of the surface, improving the access of microbes to substrates (Sylvia, 2005). During the first stage of composting, many protozoa are active too, processing particles and colloidal organic material and depredating microbial populations (Sylvia, 2005).

The populations of bacteria constitute the greatest proportion of the biological communities present in composts and are responsible for the most part of the decomposition of the organic material. Actinomycetes are common too, and give the bright and characteristic land odor in the compost. These along with fungi decompose much of the cellulose, hemicellulose and lignin present in the organic material (Sylvia, 2005). The quantification of microbial communities has shown enormous changes in the distribution of populations during the composting. Some of great participants of the composting progress from the second stage that is dominated by mesophileous microorganisms (that preferably grow between 20° C. and 40° C.), to the third stage that presents high temperatures (40° C. to 80° C.), and is dominated by thermophiles. The last stage is a gradual period of cooling, and constitutes the stage of stabilization or maturation of the compost (Sylvia, 2005).

### Effects of the Compost on the Properties of Soil

The physical properties like density, capacity of water retention, porosity and stability of aggregates are properties of soil that can be affected as a consequence of the application of composts, which are generally attributed to the increase of organic material by the addition thereof (Wallace, 1998).
a) Structure of the soil. A significantly beneficial effect of the application of compost to soil is that to improve the structure of soil increasing the integrity and stability of aggregates by the availability of the mineral fraction of soil for the microorganisms. Adding organic material increases the growth of the microbial populations (Wallace, 1998).
b) Density. Other beneficial effect to add compost to soil is the decrease of density, that increases the infiltration of water and increases the volume of pores (Rechcigl, 1995).
c) Erosion of the soil. Many investigators have confirmed the importance of the organic material in the stability of aggregates through the formation of organo-mineral complexes. The erosion of a soil depends on the strength to the wind of aggregates of soil, to the impact of the rain, or to the flow of the surface. With the addition of organic material, the amount of humus substances is increased, which serve as link agents in organo-mineral complexes. (Wallace, 1998).
d) Humidity ratio of soil. Incorporating compost to soils has resulted in an increase in the capacity of water retention, water available for plants, and pores receiving water, meanwhile there is an important decrease in the movement of the water under conditions of saturation (Rechcigl, 1995).

### Effects on the Soil Chemistry

The addition of organic material to soils significantly increases the cationic exchange capacity of soil. This capacity is primordial in the nutrition of the plants and in the handling of the fertility of soil, certainly constitutes a temporary container for cations and is considered as an indicator of nutritional capacity of the soil (Wallace 1998). The additions of compost can also alter the pH of the soil, which affects the viability of ions and its absorption by the plants. An increase in the pH offers a strong absorption of particles of the soil, in some cases, the precipitation of Cd, Mn, Pb and Zn, and other metals, which allows a low accumulation of metals in the vegetal tissues (Wallace, 1998).

### Substrates

A substrate is all solid material other than the soil, natural, of synthesis or residual, mineral or organic, that when is placed in a container, in a pure form or mixed, allows the anchorage of the radicle system of the plant, therefore a support role for the plant. The substrate can or not take part in the process of the mineral nutrition of the plant. In horticulture the substrates have been used for (García, 2002):
The growth of seeds.
The propagation of plants.
The production of vegetables.
The production of ornamental plants.

The properties substrates should have to obtain a good yield in the growth of the plants are:

### a) Physical Properties

Porosity

Porosity is a measurement of the total volume of the substrate that both solid particles and the spaces between them containing air or water may occupy. This value is usually expressed in percentage, that is a substrate with 50% of porosity that is a half of solid particles and a half of the pore space. Its optimal value does not must be inferior to 80-85%, although the substrates with lesser porosity can be advantageously used in particular conditions (Plaster, 2003).

Porosity must be great, since having a more quantity of pores, these are in contact with the open space, which induces an exchange of fluids with the open space and therefore serves as storage for the root. The size and quantity of pores conditions the aeration and water retention of the substrate (Plaster, 2003).

Density

A substrate has a particle density and an apparent density. The density is the ratio between the weight and the unit of volume. The density of a particle is determined by the weight of a solid particle of the substrate divided between the volume of the solid particle of the substrate. In order to obtain a solid particle, one must compress the sample of substrate until eliminate the space between the pores, that is occupied by air or water. The apparent density indicates the total space occupied by solid components besides the space of pores. The apparent density indirectly indicates the porosity of substrate and its facility of transporting and handling (Sylvia, 1999).

Density has a relative interest. Its value varies according to the material to be about and often fluctuates between 2.5-3 g/ml for the most substrates of mineral origin. The low values of apparent density are preferred (0.7-0.1 g/ml) and that may guarantee a certain consistency of the structure (Sylvia, 1999).

Structure

It can be granulated like the most mineral or fibrillar substrates. The first has not a stable form, easily coupling itself to the form of container, meanwhile the second depends on the characteristics of fibers. If they are fixed by some type of cementation material, may conserve rigid forms and do not adapt themselves to the container but have certain facility of volume change and consistency when go through dry to wet (Wallace, 1998).

Granulometry

The particle distribution affects two important aspects of substrates: the area of the internal surface and the number and size of the space of the pores. The area of the internal surface is the total area of the surface of all particles in the substrate. Then the substrates with many small particles have a bigger internal surface area (Sylvia, 1999). The internal surface area is important because the reactions take place on the surface of particles of the soil. If the particles are very big the most of water would drain very quickly, having a better aeration. Following the rule of size of the particles, a substrate with small particles retains more water because there is more area of internal surface for the water may be adhered (Sylvia, 1999).

The size and number of pores depends on the size of particle, that is that with big particles there are big pores and with small particles there are small pores. The substrates with big particles drain water quickly, and as the water is drained, the spaces are filled with air. And the particles with small size tend to retain water. Both sizes are important because the substrate needs micropores to retain the water and macropores for the air (Sylvia, 1999).

Water Retention

The water a substrate may retain and that is viable for the plants are two different characteristics, since only the portion of water of the substrate between the field capacity and the point of exhaustion is available for the plants. The water a substrate may retain and that is viable for the plants are established in the texture of the substrate (Plaster, 2003).

For example substrates with very big particles, their internal surface area is very small to retain water films. In addition to this, the pores are too big and so much of the volume of each pore is so far from the surface of particles to retain water against the gravity. On the contrary, in substrates with median to small particles that consequently have smaller pores and their internal surface area is greater, have a great capacity of water retention, but not great capacity of retention of the available water. The substrates with a mixture of both particles have the greatest capacity of retention of the available water (Plaster, 2003).

### b) Chemical Properties

The chemical reactivity of a substrate is defined as the transference of material between the substrate and the nutritious solution that feeds plants through the roots (Kohnke, 1995). This transference is reciprocal between the substrate and the solution of nutrients and may be due to reactions of different nature:

Chemical

These are due to dissolution and hydrolysis of the substrates themselves and can bring about:
1. Phytotoxic effects by liberation of H⁺ and OH⁻ ions and certain metallic ions like the Co⁺².
2. Deficiency effects due to alkaline hydrolysis of some substrates that brings about an increase of pH and the precipitation of phosphorus and some microelements.
3. Osmotic effects brought about by an excess of soluble salts and the consequent decrease in the water absorption by the plant.

Physico-Chemical

These are reactions of exchange of ions. They take place in substrates with contents in organic matter or those of clay origin that is, those wherein there is a certain ability of cationic exchange (A.C.E). These reactions bring about modifications in the pH and in the chemical composition of the nutritious solution thereby the control of nutrition of the plant is difficult (Kohnke, 1995).

Biochemical

These are reactions producing biodegradation of the materials composing the substrate. They take place mainly in materials of organic origin, destroying the structure and varying their physical properties. This biodegradation releases CO₂ and other mineral elements by destruction of the organic material. The chemical activity provides additional elements to nutritious solution by process of hydrolysis or solubility. If these are toxic, the substrate does not serve and it must be put aside, even though these nutritious elements are useful, they obstruct the balance of the solution when place above their incorporation an extra-contribution therewith it will rely on, and said contribution has not guarantee of quantitative continuity (temperature, exhaustion, etc). The chemical process also prejudice the structure of the substrate, changing its initial physical properties (Kohnke, 1995).

### Organic Substrate

The organic substrate more used in the industry of greenhouses is prepared with turf of Sphangum, due to its low variation of degradation and to its high physical and chemical stability (Benito 2005). The turf of Sphagnum is a bryophyte which is accumulated in the marshy peat bogs, that forms a very acid mass, with a pH of approximately 4.0, quite oxygenated and with a low content of nutritious minerals. Through the years, the sphagnums are accumulated in the peat bog and form a moss due to natural conditions of marshy soils, this turf messes up very slowly and, may form a 1 to 6 meters thickness mattress along thousand years periods. The peat bogs are constituted of 92% of water. Foreseen to harvest, ditches are digged within and around the peat bog to drain the water around the moss. The nonuseful vegetation is eliminated and stumps, big roots and other residues are eliminated from the surface of peat bog too. The turf of Sphagnum with more than 15 cm thickness is harvested, it is left to dry in ambient air, and then, by the use of a vacuum cleaner, the upper part with more than 5 cm thickness is harvested. The high capacity of cationico exchange of turf is unfavorable for the vegetal nutrition, since it presents a 3.5-4.0 pH level.

The high cost of commercial turf, along with the exhaustion of this nonrenewable resource and the consequent environmental deterioration, has supported the generation of products able to replace this substrate, total or partially, (Guérin, 2001).

Another substrate of industrial importance is the coconut fiber. In recent years, said substrate has achieved to compete with the turf of Sphangum. The product has a water retention capacity up to 3 or 4 times its weight, it has a slightly acid pH (6.3-6.5) and its porosity is quite good. However, its availability is scarce in regions remote from the sites of production, besides that an exhaustive washing is necessary to its use, since its stabilization is based on a salt-rich product that allows its stabilization. Another problem of coconut fiber is that its process of production does not prevent the contamination by pathogenic microorganisms, or seeds of weeds (Prince, 2000; Wilson, 2001).

### Composts as Conditioners

To develop alternative organic substrates at low costs and rich in nutrients can eliminate not only the environmental impact, but also can help to reduce the costs associated to fertilization, low irrigation volumes and eliminate the costs associated to the suppression operations of pathogens (Benito, 2005).

Composts have the advantage to be produced at low costs (Wilson, 2001; Pérez, 2006), they can act like an effective cover of nutrients increasing the concentration thereof in the soil, improving the water retention availability and capacity and weed suppression. The application of composts guarantees the permanence of nutrients in the soil and assures the maintained production of agricultural products of food concern (García, 2002).

Several authors have reported the use of composts as covering grounds with different compositions. Composts have been developed based on agro-industrial residues or harvests, municipal solids, and residues of gardening, among others. The application of composts has improved the physical properties of soil, as well as the quality of soil nutrients, which has allowed to obtain plants of better quality in cultures of commercial importance (Stabnikova, 2005).

Many works have been focused to the use of composts produced from municipal muds, due to their high content of nitrogen, phosphorus and trace elements. The composts of this type were tested as conditioners in substrates for different vegetables culture. The results of said studies indicate that these composts seem to be a good supplement of nutrients, as well as evident enhancers of physical properties of the subsoil. The application of said composts did not affect the levels of germination efficiency. The contents of heavy metals in plants were below the toxic levels. (Warman, 1996; Stabnikova, 2005; Perez-Murcia, 2006).

Starting materials of different origins have been used for the formulation of composts. They are found among them: residues of gardening derived from the reaping of grass (Bodin, 2005; Benito, 2005); residues of the wood industry (Hernández 2005); agro-industrial harvests like muds of process of malting (Garcia 2002), settlings of wine production, and stems of grapes (Bertrán, 2004); short paper fibers (Ekinci, 2000) and residues of the clarification of paper pulp (Simone, 2003); waste waters of the extraction of olive oil (Paredes, 2005), to mention some of them.

In some of referred works, the composts were tested as additives in substrates for the culture of plants of agricultural or ornamental concern. Likewise, physical and chemical properties were determined with the purpose to determine the maturity and quality of compost, and germination assays were carried out. In the most part of the studies, the increases in the weight of plants, in the number and weight of leaves, the number of buds, in the total height of plants, and in the thickening of stems, were reported.

In all studies, the composts and their application in agricultural and horticultural production have only be focused on their use as an additive with the main objective to be a biofertilizer (not more than 50% in the ratio of substrate to compost) for a substrate, one of barriers for the production of composts on an industrial scale to obtain as final product a substrate for greenhouses to be elaborated from wastes and being a product consistent and predictable in quality (Guérin, 2001; Simone, 2003; Hernández, 2005). To maintain a compost of uniform quality is a particular problem when the resource it comes from has a great variability and a high amount of organic material. That is why the operations of the process of composting must be optimized to guarantee the efficiency of transformation of wastes, and to assure an uniform quality of the final product (Simone, 2003).

One of the main problems carried by the excessive use of composts is associated to the intoxication of the plants due to high contents of salts and the consequent accumulation of heavy metals in the culture grounds treated with composts. Said accumulation can risk the health of humans and cattle consuming these plants, mainly when composts made with municipal wastes have been used (Soumaré, 2003).

One of main characteristics of composts, that has obstructed their widespread use as agricultural substrates is their high density. Composts of typically high density and low porosity are compacted after irrigation, and can generate breakings in roots and stems, besides to limit the gaseous interchange and commit the good drainage of the soil.

That is why there is the need of a process of composting for the profit of organic materials consisting in the generation of substrates for horticulture, for intensive agricultural application in the field, for agricultural and forestal greenhouses and nurseries, therewith it is possible to obtain a product that allows the substitution of the main substrate of use in agroforestry production systems, turf or peat moss.

The use of wastes of the sugar industry as compost for the formulation of a substrate as enhancer of the soil in a system of tomatoes production was studied by Stofella and Graetz (2002). Muds of the clarification of juice of sugar cane or press mud were mixed with water and pumped from mills towards fields, allowing them to stay in marshes for more than a year. The final product was a colored compost with the appearance of a high calcium soil. In some systems of containers in the production of seeds of citruses this compost has been a partial substitute of turf. For tomatoe plants, the containers that were formulated with compost had plants with heavier buds, thicker greater total height, and commercially, a higher number of bigger and heavy fruits at an early age than containers without compost.

In 2006, Ram and collaborators developed a study on the optimization of application of water and nitrogen in mint cultures (Mentha arvensis L.), through the wastes of the straw of sugar cane in a sandy-clay soil of a subtropical semi-arid climate. Mint is an important culture for industry. As essence, mentol is widely used as flavouring in pharmacy, cosmetics, and alimentary industry. Mint cultures favorably respond to fertilizers with high levels of nitrogen. The agricultural yields oscillate between 150 and 200 kg/ha. On the basis of growth patterns of the mint, and under different regimes of fertilization with nitrogen, the results of the study allowed to conclude that the greatest growth was registered in batch added with straw of sugar cane, under conditions of a 134 kg/ha irrigation radius in 16 applications. In those conditions, the nitrogen requirement for the production of 200 kg of mint by hectare was covered with the application of harvests of cane.

As above mentioned, the substitution of turf has been sought due to its high cost of production, and that its harvest had negatively impacted on the atmosphere, causing an evident environmental deterioration. In the process described the U.S. patent application No. 20050274074, the purpose is to generate an appropriate substrate as well as for the culture of plants and for the production of fungi. The resource contains muds of the milling of sugar cane, coconut fiber, and materials not derived from Sphagnum. It should be understood that the term “not derived from Sphagnum” includes any material used in turf not derived from Sphagnum moss or peat moss. Materials of that type can be anyone derived from trees or shrubs, and the other material containing the substrate is coconut fiber which is commonly known as coconut turf, this is the most fibrous part of the crust of coconut.

The muds of the milling of sugar cane refer to washed material including washings of cane, slime, impurities of cane juice and fine bagasse.

Both, the not derived from Sphagnum materials and the muds of the milling of sugar cane, when used individually for the growth of plants or as enhancers of the soil, were useful for the culture of plants and the proliferation of fungi. However, in the referred investigation, it was determined that mixing both materials (those not derived from Sphagnum and the muds of milling of sugar cane), a superior material is obtained, of better quality for its use as culture resource for the plants or for the improvement of the soil for the agricultural production.

In the described process, the relative concentrations of the components not derived from Sphagnum and the muds of milling of sugar cane, were optimized in order to obtain the desirable properties of the substrate. The formulation was adjusted to arrive to optimal levels of water retention, aeration, pH, content of salts and levels of nutrients. For example, the materials not derived from Sphagnum have undesirable properties like a low pH, low levels of nutrients and too much porosity. In contrast to, the muds of milling of sugar cane have a high content of salts, low water retention, presence of pathogenic microorganisms, or susceptibility to the growth thereof. In the case of the U.S. patent application No 200502844302, the generation of a substrate for the culture of plants is described. The inventors generated a mixture for flowerpots only based on sawdust of pines, or mixed with organic materials of wastes. The material is ground by a continuous process, and submerged in hot water containing a chemical additive for treatment. The mixture is partially drained until losing 15-25% of its weight. The retention capacity of the final formulation is near to 50% in weight.

In the patent application No. WO1994022790, a method to produce a substitute for peat moss is described. The invention is related to formulations of cellulosic starting materials, an inoculate with degrading microorganisms of this material in the form of ammonium-generating bacteria, besides degrading bacteria of lignocelluloses, fertilizers, and municipal wastes or similar wastes. The mixed material is treated with steam.

The patent application No. WO2003002638 A1 refers to a treatment of vegetal fibers and straw with formaldehyde, and starches, for the generation of a mixture. The mixture is treated with high pressures of steam (300-450 kg/cm²) and 120 to 180° C.

The products generated in the referred inventions consist of useful materials for the production of substrates of easy degradation and good characteristics of support. Nevertheless, in this generated product, the materials are treated with chemical disinfectant products, or with high steam and temperature, which requires a source of thermal energy and advanced-technology reactors of continuous operation.

In general, in rural locations, in operations near to production of straws, bagasses and lignocellulosic agroindustrial residues or harvests, a suitable profit from these residues is not carried out. The handling and treatment of agricultural by-products often represent a very high cost when they are developed with advanced-technology systems, in automated reactors insulated from rain or drought. The necessary investment for this type of equipment, that processes thousands tons of material, costs in the order of millions dollars. Therefore, and with the purpose to low the investment costs, it is necessary the process of said materials be carried out in open air operations, or under cover, with semi-mechanized systems, of blenders or mixers type. In those that it is sought that during the composting, the metabolic heat generated by biodegradation of celluloses allows the production of substrates with suitable characteristics for the production of plants, free of phytopathogenic microorganisms, and seeds of weeds. Wherein the investments in equipment and infrastructure be smaller, more economic, and suitable to the process of great amounts of lignocellulosic material in difficult-access rural zones.

### Wastes of the Sugar Industry in Mexico

In Mexico, two of agro-industrial residues generated in a higher volume are those of corn and sugar cane. In this country the corn production takes place in a very dispersed way, which causes enormous difficulties in its harvest and use. With comparison to, sugar cane is processed in centralized places, and the associated costs of storing are minimum, that is why the present invention focuses to these resources.

Nowadays, 58 sugar mills operate in Mexico (22 of these in the State of Veracruz). Altogether, the Mexican sugar mills produced about 5.8 millions tons of sugar, during the 2004/2005 crushing season. The total of crushed cane was 50.9 millions tons (2005/2006 Infozafra). Sugar mills produce a great amount of residues, which not always are useful, and therefore cause pollution in subterranean aquifers and superficial sources of water for the human consumption. If the environmental impact of said residues is considered, the importance to develop a basic investigation and applied on the field of processing of solid agro-industrial residues can be deduced, the manufacture of mature composts and fermented fertilizers can contribute to the improvement of the quality of soil and to the conservation of a healthy environment.

The effect of the application of stubbles of sugar cane in a vertisol soil on the physical and chemical properties of soil and the yields of sugar cane in agricultural production was investigated in the State of Tabasco (Sánchez, 2003). After two years of recycling stubbles of harvest in vertisol soil cultivated with cane, the soil wars evaluated in order to determine the effect of stubbles on its physical and chemical properties as well as the yield of culture. The applied formulations were T1 burning of stubbles (witness), T2 stubbles placed in bands on the central trenches of the parcel and T3 chopped and scattered stubbles in the parcel. The studied variables were organic material, total nitrogen, phosphorus, pH, apparent density, residual humidity and yields of harvest. It was concluded that in the culture of sugar cane, the recycling of stubble (T2 and T3) does not promote changes in the variables the study in a two years period. In earlier ages, the treatment T2 reflected better edafic indicators than T1 and T3, although the residual humidity decreased in harvests immediately later to the contribution of stubbles.

In the State of Veracruz, in Mexico, studies associated to composting of by-products of sugar cane have been developed. In the central sugar mill Motzorongo, the microbiology of a compost formulated with by-products of cane and with the associated process of composting was characterized. The derivatives of sugar production that can be used for the elaboration of compost are bagacillo, bagasse and press mud (Rosas-Morales, 2003; Meunchang et al, 2095).

Bagacillo is a fibrous lignocellulosic residue that is obtained from the last milling of sugar process and is formed by an heterogenous set of fibers whose length is between 1 and 25 mm. It comes from the mixture of four different portions, morphologically identifiable, of the stem of sugar cane:
The epidermis, which constitutes 5% of the bagacillo, corresponds to the cuticle of sugar cane, and is formed by waxes that constitute the main protection of stem against acids and pathogens. The epidermis acts as a waterproofing of the internal sugar to the outside.

Chemically, this fraction is made by those called “extractable” of bagacillo:
On the other hand, the crust contributes to the rigidity and hardness of stem, and is made by fibers of determinated size and diameter which constitute the majority fraction of bagacillo.
The parenchyma (30% of bagacillo) is the tissue responsible for the storage of the sugary juices.
The fibrovascular bundles (15% of bagacillo) immersed in the parenchyma, are responsible for the conduction of minerals and nutrients in stem.

The crushed cane does not allow the distinction of fibers of different anatomical origins without using complex systems of macroscopic and chemical analyzes of same. In general, two typical fractions are known, the fiber constituted by fibers of the crust and parenchyma, and the marrow, constituted by fibrovascular bundles, epidermic fibers and small particles of soil (Rosas-Morales 2003).

The chemical composition of bagacillo allows to know that between 41 and 44% is cellulose, a polymer of glucose residues attached by bonds beta 1-4; the hemicelluloses, that constitute between 25 and 27%, are given mainly by xylans and mannans. Finally, lignin that is a compound which constitutes between 20 and 22% of bagacillo is formed by complex polymers of phenolic nature (Rosas-Morales, 2003).

The press mud, another of agro-industrial derivatives of the sugar cane process, is a mud that is removed during the clarification of cane juice. The press mud, also known as filtration mud, is obtained by sedimentation of the colloidal material contained in juice, and is obtained by precipitation of the insoluble solids from using of alkalizers that flocculate by formation of insoluble salts (calcium phosphates fundamentally) (Rosas-Morales, 2003). Instead, the bagasse comprises both the bagacillo or marrow, and the crust, or long fiber.

The press mud or mud of filtration, is recovered as a mud with very high humidity. Its water content is between 75 and 77%, and the corresponding dry material constitutes between 23 and 27% (Rosas-Morales, 2003).

The press mud is made up of a rich mixture of sources of nitrogen and carbon and simultaneously phosphatized minerals and other types (Table 1).

The amount of press mud obtained in percentage in relation to cane, and its composition, greatly varies with respect to the different localities of production, depending on the variety of processed cane, the efficiency of milling, and the method of clarification, among other parameters (Rosas-Morales, 2003).

In a study of the process of composting of press mud and bagacillo as starting materials at open air, it was defined that the material under composting generated both in the rain season and in the drought, presents cycles of acidification, alkalinization and an increase of temperature that oscillated between 50 and 70° C., typical of semiaerobic composting. The obtained compost had an increase of 50% in dry weight and a diminution of 50% in the C:N ratio, with respect to formulation before the composting. That the addition of bagacillo as starting material of composting had not effect on the pH, nor on other parameters in the mature compost; in fact, in starting formulations with more bagacillo this addition generated a greater porosity and better times of composting, without modifying quality of the final compost substantially. Populations of gram-positive bacteria (actinomycetes and bacilli), as well as filamentous fungi, were found in the biodegradation process. Most of these organisms are thermotolerant and lignocellulolitic, and therefore are able to degrade materials present in the pile of composting (Rosas-Morales, 2003).

Another more recent study of co-composting of press mud and integral bagasse does not refers to the microbiology of compost, but evidently establishes that the starting mixture of press mud and bagasse allows the conservation of nitrogen.

TABLE 1
Composition of press mud % in dry base

| | Standard |
|---|---|
| | Values |
| Raw protein | 12-16 |
| Extract by benzene (waxes, | 10-14 |
| resins, etc.) | |
| Ashes | 8-12 |
| Phosphate | 3-5 |
| Calcium Oxide | 2.5-5 |
| Sugars | 10-14 |
| Core or marrow | 18-25 |
| Others* | 25-35 |

*It is possible to emphasize that in “Others”, an important concentration of sugar-cane soil is probably contained, which constitutes the microbial source that functions as central inoculum of microorganisms catabolizing lignocelluloses during the composting.

The parameters used to evaluate the maturity of compost were pH, dry weight, and content of organic material and nitrogen. In conditions of variable climate, the period of composting can vary from 12 to 28 weeks depending on the season. In the rain season, the temperature of process changes, the porosity of material diminishes, and the dehydration takes place more slowly, between 24 and 28 weeks. In the drought season, the process is faster, between 12 and 20 weeks. The compost generated by this process has a very high apparent density, from 0.8 to 1.3 g/ml, a very low porosity, a low humidity retention (<60%), and a very long time of process (>12 weeks). Even though the qualities of the material as nutritional enhancer of soils is well established (Rosas-Morales, 2003; Meunchang, 2005).

The generated compost is a material useful as enhancer of agricultural soils by its nutritional quality, but in no way has suitable characteristics for its application in horticulture as a substrate in greenhouses or nurseries, even less in operations of restoration of agricultural lands damaged by drought or erosion. Furthermore its prolonged time of composting, at least 12 weeks, makes it particularly expensive.

This is why in the present invention the traditional process of composting of agro-industrial residues, more preferably of lignocellulosic residues, even more preferably of fibrous lignocellulosic residues, such as residues of sugar mills, corn, agave, straws of gramineous plants and grain husks such as rice and barley was modified. Specifically of bagacillo of cane, bagasse of cane, press mud or mud of filtration. Through this new process of semi-aerobic mechanized composting, the material is mixed, and degraded during 7 to 8 weeks, reducing largely the time of composting. During the process, the carbon is depressed, and therefore the C:N ratio, the material is dehydrated, and nitrogenous nutrients are concentrated. Furthermore a mature substrate is generated, but of great porosity, low density and very high humidity retention, that enables its use as a sole substrate in greenhouses and nurseries, and its application as dual humectant agent and nutritional enhancer, in agricultural and forest soils.

### SUMMARY

The present invention contributes with an improved process of composting for the production of a low-density humectant substrate (LDHS), which allows a substantial improvement in the quality of the final product. Through this new process, derived from an intensive experimentation, a material free of pathogens (as well as traditional or typical compost), with very low density (0.2 to 0.4 g/ml), high porosity, and very high water retention (>90%), fully applicable for its use as substrate of production in horticulture, forestal in nurseries, as well as humectant agent and enhancer of soils in the agricultural field and eroded soils, is generated during 7 to 8 weeks.

The new process also provides a low-density humectant substrate (LDHS) produced from agro-industrial residues, more preferably of lignocellulosic residues, even more preferably of fibrous lignocellulosic residues, such as residues of sugar mills, corn, agave, straws of gramineous plants and husks of grains such as rice and barley. Specifically of bagacillo of cane, bagasse, press mud or mud of filtration, by a controlled process of composting, by fed batch.

Furthermore, the process of the present invention provides a LDHS with characteristics similar to those of peat moss or turf, and those of other fibers used in agriculture and horticulture, that not only has utility in agricultural and forestal greenhouses and nurseries, but also as humectant agent for restoration and recovery of soils and for the establishment of agricultural and forestal plantations of great success and productivity.

Likewise, the present invention provides a product useful for its use in greenhouses and nurseries, either as substitute or complement of other products (peat moss, coconut fiber, polyethylene covers), or in mixtures with natural and synthetic substrates for agricultural and forestal production.

As well the present invention provides a process of production of a substrate, which guarantees a reproducible quality, physico-chemical and biological stability that allows the optimal germination of seeds.

The purpose of the present invention is also the profit of agro industrial residues, even more preferable of lignocellulosic residues, even more preferable of fibrous lignocellulosic residues, such as residues of sugar mills, corn, agave, straws of gramineous plants and grain husks such as rice and barley. Specifically of bagacillo of cane, bagasse, press mud or mud of filtration or other equivalent harvests of lignocellulosic materials, which allows a better alternative of profit of wastes.

### DESCRIPTION OF THE INVENTION

The process and product generated under the present invention are based on an improved process of composting from residual materials like agro-industrial residues or harvests, more preferable of lignocellulosic residues, even more preferable of fibrous lignocellulosic residues, such as residues of sugar mills, corn, agave, straws of gramineous plants and grain husks such as rice and barley. Specifically of bagacillo of cane, bagasse, press mud or mud of filtration.

The main contribution is stablished in the application, in different stages of the process of composting for the obtention of low density humectant substrate, of lignocellulosic materials in a controlled and defined way that were stablished by experimentation. The addition materials are added with the intent to obtain a material with greater porosity, lower density and better capacity of water retention. The used addition materials can be: bagacillo of cane, bagasse of cane, bagasse of agave or straws of corn, gramineous plants, and grain husks such as rice and barley, any fibrous lignocellulosic residue in general. Under this improvement the process of composting, becomes a system of controlled solid fermentation, of fed batch.

The used system of composting is that of windrows or semi-static biopiles. The first step of the process consists in the cleaning of the composting area, that is carried out with the aid of a tractor. Later, the experimentation area is delimited, in order to mark the position of biopiles or windrows (in English language: windrows), drawing rectangles in the soil according to dimensions of the well-known technique of 3 m×2.5 m (7.5 m²), to generate biopiles of 1.4 m of height, or for piles of industrial scale from 12 to 15 m×2.5 m (30 to 37.5 m²) to form piles of 3 m of height.

With an starting composition of materials constituted by bagacillo of cane, bagasse, press mud or mud of filtration. In a proportion of starting materials of press mud and bagacillo of 86:1, 43:1, 10:1. The material is deposited, in the open air, on an absorbent litter of bagasses or straws about of 5-15 centimeters of thickness, in order to avoid the draining and loss of juices contained in press mud. The material is homogenized with a mechanical composter, for the purposes to distribute the starting materials uniformly, besides the use of composter allows the distribution of oxygen, which is indispensable in the first weeks to increase the aerobic microorganisms activity present in the compost.

After this first mixing, the material to be composted is left to repose during a week, that is without turnings. After a week to start the composting, the controlled feeding of fresh lignocelulosas begins, by means of the addition of a load of bagacillo, of 1-3.5% weight with respect to the amount of starting press mud. The material is intensely homogenized with the composter and is left to repose during one more week, that is without turning by the composter. After that first cycle, the temperature of the pile is typically between 50 and 60° C. even in repose. 5 more cycles of additions of bagacillo are repeated, corresponding to the following 5 weeks.

The additional feedings consist of the same weight of bagacillo that of the first event of feeding. During that period of biodegradation by the process of fed batch, the material under composting generates temperatures associated to systems of bioconversion of thermophiles, between 65 and 85° C. typically. The cycles of addition of lignocelluloses by fed batch help the pH to be neutral, without drastic changes. From the second cycle, there is a quick reduction of volatile biodegradable solids and therefore of the production of disagreeable odors. In the subsequent week to the last cycle of addition of bagacillo, the material is left to repose during a week, without turning by the composter.

The products of composting are stabilized by dispersion of still warm material, with the intent to low their temperature, and to allow the evaporation of the excessive humidity. This drying process consists to disperse the complete pile of glitters, which are exposed to the sun during 2 or 3 weeks, until reducing 30% of total humidity of compost. While stays exposed to the sun, the compost must be turned around and mixed every 5 days, with the purpose to have a homogenous drying. Finally a mature compost, with good characteristics of texture, and excellent both physicochemical and biological properties is obtained. In said process, phytotoxic compounds have been metabolized and the microbial pathogens have been eliminated from the plants, which do not typically support temperatures of more than 60° C., and even less for long

The low density humectant substrate that is the final product obtained from the improved system of composting is generated, during 7 to 8 weeks, as a material free of pathogens (like traditional or typical compost), of very low density (0.2 to 0.4 g/ml), high porosity, and very high water retention (>90%). It contrasts with mature composts, obtained from batch operations, not derived from controlled feeding of materials during the process. This difference is due that the physicochemical characteristics of typical composts are not appropriate to its use as humectant substrates, since their high density and prevailing granulometry of very fine particles make them more similar to a superficial soil than to an humectant substrate. Said characteristics limit their use as substrates for nurseries, as supports in greenhouses, as well as their application in agricultural plantations, in the form of humectant covers.

### Analytical Methods

The materials under composting were sampled by the use of a cylindrical punch with a 15 cm diameter and a meter of length. The samples were of 1000 g of representative material of all levels (from the center to the surface) of the material under composting.

The compost samples were stored for periods not greater than 2 weeks under 4° C. of refrigeration until their analysis.

In laboratory, the samples were analyzed with respect to granulometric profiles, pH, apparent density, water retention, humidity and porosity. The samples with similar characteristics to peat moss were selected for the germination assay.

### Humidity by the Gravimetric Method

The humidity and total solids were determined with the gravimetric method. About 10 g of an humid sample were placed in Petri dishes, and the exact weight was determined with the help of a Voyager Ohaus analytical scale. The weight of each sample was registered and then deposited in a stove to 90° C. The weight was monitored every 24 hours until it was remained constant and established as final weight. Later, the percentage of solids and humidity present in composts was determined (Valdés, 2005).

Then, the content of humidity and total solids was calculated as the percentage of weight of the humid sample.

Humidity(H)=(Weight of the humid compost)−(Weight of the Dry Compost)

Percentage of Humidity=(H*100)/10

Percentage of total solids=100−Percentage of Humidity

pH

In this test, the concentration of hydrogen ions is determinated in a solution of compost. The pH was determined diluting a part of compost in 1:2 proportion (10 g of compost and 20 ml of water). The sample was homogenized by vortex and then was left to repose during 30 minutes. After the repose, the sample was vigorously shaken and the pH was determined by measure with an Orion potentiometer, 410a model. The samples of sole press mud (samples 3 and 4) without addition of bagacillo were included in the pH determination, as well as samples of turf and coconut fiber (Valdés, 2005).

### Apparent Density

The apparent density is the mass of a substrate by unit of volume expressed as g/cm³. Once the apparent density is known, the measurement of the mass of the substrate, the percentage or volume can be expressed interchangeably or in absolute terms (Okalebo, 1993; Plaster, 2003).

The apparent density of the samples was determined using a 1 L test tube wherein a sample of 200 grams was placed. The volume occupied by the sample was determined and then the density was calculated.

### Water Retention

It is defined as the maximum amount of water, that after the free drainage, can retain a certain substrate. It is estimated after a substrate is saturated with water, and the drainage has been allowed without leaving its humidity be eliminated by evaporation (Okalebo, 1993).

For the determination of the capacity of water retention, 5 to 10 g of the sample of substrate are weighted in a Petri box. This is placed in the stove to 90° C. until obtaining the constant weight. A disc of paper filter is saturated with water, its weight is registered and is placed in a funnel. The dry sample is spilled in the funnel and is weighted, water is added to the sample until saturate it, and is left to drain until the dripping stops. Once the dripping stops, the final weight is registered (Okalebo, 1993).

For the calculation of the capacity of water retention:

(U)Unity=Weight of the paper filter+compost

Weight of the U with saturated soil=Weight of the humid paper filter+weight of the dry sample

Retained Water=Weight of the paper filter with the sample saturated with water−weight of the paper filter with the dry sample.

Capacity of Water Retention=(Retained water*100)/10

### Granulometry

In order to measure the distribution of the size of particles, the different compost samples were dried at ambient temperature during three days, 100 g of each sample were taken and passed through four sieves of different measures. The residue remaining in each sieve was weighted. The pore sizes of the used sieves were 1.98, 0.5, 0.025, and 0.005 millimeters (Benito, 2005).

### Assay of Germination

In order to guarantee the utility of samples of substrate as culture resource in greenhouse, a test of germination was carried out using seeds of grass and tomato. 20 g of sample were weighted, the seeds were washed with chlorine at 10% and rinsed with deionized, sterilized water. Then, they were added to the 20 g of sample of substrate placed in a glass Petri plate. Constantly maintaining the humidity to saturation for all the assays, the plates stayed during 7 days in a camera of vegetal growth with 12 hours periods of light and at constant temperature of 25° C.

### Porosity

The porosity was determined drying 1 kg of sample of each substrate in a stove at 90° C. for 72 hours, until obtain a constant weight. Then, the dry sample is deposited in a test tube until reaching 500 ml and with the help of an analytical Voyager Ohaus balance, the weight of each sample is registered. The following step is to take the sample from the test tube and place it in a tray with water until it is completely saturated. Later, the sample is retired from the tray and is left to drain until the dripping stops, finally the weight of the drained sample is registered (Plaster, 2005).

### Calculation of the Percentage of Porosity

Percentage of Porosity=(Humid Weight−Dry Weight/Volume (Humid Weight))*100

### Example 1

For this test, windrows of 3.0 tons of the starting material were used. The system of composting for the substrate was by semi-static piles with aeration by periodical homogenization. In each homogenization, a composter that worked to each windrow was used during 20 minutes. The homogenization, besides to incorporate materials uniformly, enabled inclusion of air in the compost.

For the starting materials, different formulations were evaluated, which differ by the proportion of bagacillo and press mud contained therein. Different regimes of addition of bagacillo in the formulations of fed batches were tested. The design of the experiment is submitted in table 2.

Each treatment, with its duplicate, was developed according to table 2. The amount of bagacillo was added to the windrow according to formulations. After starting the process, the bagacillo was added in different proportions every week. The additions continued until reaching a 10 weeks period. After the corresponding addition of bagacillo, the compost was homogenized and a sample from each windrow was taken for analysis in laboratory.

TABLE 2
Composition of the piles for process of composting

| | Ratio of | | | | | | | | | | | | | |
|---|---|---|---|---|---|---|---|---|---|---|---|---|---|---|
| | Press mud: | WEEKS | | | | | | | | | | | | |
| No | Bagacillo | 1 | 2 | 3 | 4 | 5 | 6 | 7 | 8 | 9 | 10 | 11 | 16 | 20 |
| 1 | 86:1 | & M | & M | M | M | & M | & M | M | M | & M | & M | M | M | M |
| 2 | 86:1 | & M | & M | M | M | & M | & M | M | M | & M | & M | M | M | M |
| 3 | 1:0 | M | M | M | M | M | M | M | M | M | M | M | M | M |
| 4 | 1:0 | M | M | M | M | M | M | M | M | M | M | M | M | M |
| 5 | 43:1 | && | && | && | && | && | && | && | && | && | && | M | M | M |
| | | M | M | M | M | M | M | M | M | M | M | | | |
| 6 | 43:1 | && | && | && | && | && | && | && | && | && | && | M | M | M |
| | | M | M | M | M | M | M | M | M | M | M | | | |
| 7 | 43:1 | M | M | && | && | M | M | && | && | M | M | M | M | M |
| | | | | M | M | | | M | M | | | | | |
| 8 | 43:1 | M | M | && | && | M | M | && | && | M | M | M | M | M |
| | | | | M | M | | | M | M | | | | | |
| 9 | 10:1 | M | M | M | M | M | M | M | M | M | M | M | M | M |
| 10 | 10:1 | M | M | M | M | M | M | M | M | M | M | M | M | M |

& addition of 1.7% of bagacillo with respect to the initial weight of press mud
&& addition of 2.3% of bagacillo with respect to the initial weight of press mud
M sampling

After the reposing period, the compost should have a drying stage. The drying stage consisted of dispersing the complete pile of compost in 50-60 cm litters, which is exposed to the sun during 10-15 days, until reducing 30% of total humidity of the compost. Meanwhile the exposition to the sun is maintained, the compost must be turned around and mixed every 5 days, with the purpose to have an homogenous drying.

### Example 2

To carry out this example, it was chosen a formulation whose substrate presented the physical characteristics similar to those of peat moss and corresponding to characteristics of a substrate ideal for horticulture, and that besides exhibited extraordinary results for the germination essay. The comparison of the characteristics of peat moss and the substrate of the formulation 5, 6 of table 2 is submitted in table 11.

For this example it was used a formulation for composting on industrial scale which was of 100 tons whose proportion of the starting material was 43:1 of press mud:bagacillo. 2.3% of bagacillo was added to this windrow with respect to the weight of the initial press mud.

Simultaneously, two control windrows were established, with a proportion of 43:1 and 30:1 of press mud:bagacillo. With additions of 2.3% and 3.3% of bagacillo with respect to the weight of the initial press mud.

The experimental design of these tests is submitted in table 3. In this experiment, the additions of bagacillo were interrupted in the seventh week, that corresponds to the stage of better characteristics of substrate, defined by the analysis of materials in the experiment of example 1 (see Table 2).

To the samples weekly obtained from biopile or windrow of 100 tons, as well as to other two windrows, all the physical and chemical parameters described in the methodology were determined.

The laboratory analysis of the low density humectant substrate allowed to determine its quality, in comparison with characteristics of an organic substrate peat moss or a turf of Sphangum.

TABLE 3
Composition of the windrows 1A on industrial
scale, controls 2A and 3A, sampling weeks and
addition of bagacillo according to design.

| | Ratio of | | | | | | | |
|---|---|---|---|---|---|---|---|---|
| | Press mud: | | | | | | | |
| | bagacillo | 1 | 2 | 3 | 4 | 5 | 6 | 7 |
| 1A | 43:1 | & M | & M | & M | & M | & M | & M | M |
| 2A | 43:1 | & M | & M | & M | & M | & M | & M | M |
| 3A | 30:1 | && | && | && | && | && | && | M |
| | | M | M | M | M | M | M | |

& addition of 2.3% bagacillo with respect to the initial weight of press mud
&& addition of 3.3% bagacillo with respect to the initial weight of press mud
M sampling

### Characterization of Low-Density Humectant Substrate (LDHS)

The physicochemical changes of the press mud subject to composting were evaluated in both examples, by the effect of the addition of bagacillo, and the frequency of the addition of same (Tables 2 and 3). The samples of different windrows were evaluated with respect to the established parameters: humidity, granulometry, pH, water retention, apparent density, porosity, percentage of germination. And for comparative purposes, the commercial peat moss was used as control.

In table 4, the results of the content of humidity present in composts in the different formulations are submitted, the content of humidity is a suitable indicator of the quality of composts as substrate.

A substrate that conserves sufficient humidity allows to low the irrigation costs. A suitable content of humidity favors the germination of seeds and the growth of cultures, an excess of same can bring about deficiency of nutrients and development of fungous diseases.

In the specific case of peat moss, the substrate of great use in greenhouses and nurseries, whose content of humidity is about 45.5%. The compost samples of formulations 5, 6 and 7, 8, corresponding to the fourth and seventh weeks, have an humidity content similar to peat moss, about 40-43%. The control formulations for said experiment, that only contain press mud or that contain in the starting material press mud more than 300 kg of bagacillo, have contents of humidity between 50 and 60% in the same weeks of sampling. It is recognized that, for application in agriculture, the substrates must have an humidity content of about 50%. For all formulations in the sixteenth week they are found with an humidity lower than 35%. The substrates used for intensive agriculture, horticulture and production in greenhouses and nurseries must oscillate between 40 and 45% of total humidity. In table 5 the results corresponding to the total solid content of the composts derived from the different formulations are submitted.

The results indicate that the total solid content for peat moss is 54.5%.

Meanwhile the composts corresponding to the 5, 6 and 7, 8 formulations of the fourth and seventh weeks, have content of total solids about of 57%, the control formulations for said experiment (that only contain press mud or that contain in the starting material press mud more than 300 kg of bagacillo) have contents of solids between 50 and 40% in same weeks.

The humidity percentage of peat moss is 2.5% higher than the composts corresponding to the 5 and 6 formulations, of the seventh week, which generates an increase of 2.5% more in solids. This means that they have less than 5% of mass of water by mass of dry soil, which refers us that we can apply a system of irrigation similar to that used with peat moss.

For a mature compost it has established that the optimal pH must be neutral, from 6.7 to 7.7 (Wilson, 2001). The pH of a substrate is important because microorganisms and plants remarkable respond to the chemical changes of their environment. Most of them prefer a variation of neutral pH or near to neutral pH since the viability of many nutrients is better in this range of pH (Sylvia, 2005). For example, the actinomycetes require neutral conditions and do not tolerate the acid environments very well. Most of the fungi, that in many cases can be pathogenic, are acid tolerant (Sylvia, 2005).

Many of the elements of substrate change the result of the reactions in the soil. These reactions controlled by the pH alter the solubility of nutrients, as well as their viability.

TABLE 4
Percentage of humidity of compost samples under
different formulations with bagacillo.

| Formulation | 1 | 4 | 7 | 11 | 16 | 20 | |
|---|---|---|---|---|---|---|---|
| 1, 2 | 67 | 55 | 49 | 33 | 33 | 46 | |
| 3, 4 | 44 | 50 | 38 | 12 | 22 | 47 | |
| 5, 6 | 65 | 55 | 45 | 33 | 18 | 48 | |
| 7, 8 | 59 | 55 | 42 | 31 | 32 | 50 | |
| 9, 10 | 65 | 51 | 43 | 32 | 22 | 48 | |
| Peat moss | | | | | | | 45 |

TABLE 5
Total solids percentage of compost samples
under different formulations with bagacillo.

| Formulation | 1 | 4 | 7 | 11 | 16 | 20 | |
|---|---|---|---|---|---|---|---|
| 1, 2 | 33 | 45 | 51 | 67 | 67 | 46 | |
| 3, 4 | 56 | 50 | 62 | 88 | 78 | 47 | |
| 5, 6 | 35 | 45 | 55 | 67 | 82 | 48 | |
| 7, 8 | 41 | 45 | 58 | 69 | 68 | 50 | |
| 9, 10 | 35 | 49 | 57 | 68 | 78 | 48 | |
| | | | | | | | 55 |

It is remarkable that most of nutrients are more available in neutral pH levels. For most of cultures, a pH range of 6:0-7.0 is preferred.

The results of the change in the pH value of the press mud composts under different formulations of bagacillo are submitted in table 6.

The results indicate that, in all cases, the pH begins in levels higher than 8.0, definitely alkaline, and said values tend to neutralize, so that for the samples of the last weeks, the values of pH are near or in some cases less than 7, as in the case of the 9, 10 formulation. An ideal substrate for intensive agriculture should have values pH between 5.3 and 6.8. In the case of peat moss, the pH has a 3.8 value, which guarantees a long shelf life. However, the associate acidity can bring about the liberation of high toxic aluminum salts, which compromises the cationic exchange capacity of soil.

The results indicate that the composts corresponding to the 5 and 6 formulations, of the fourth and seventh weeks, have a pH about 7.7, which is lightly alkaline for purposes of application in intensive agriculture, but is very close to the suitable pH levels. In contrast, the 3.8 pH value of peat moss requires specific formulation to reach suitable pH levels, for its use in nurseries and greenhouses.

TABLE 6
pH of compost samples under different
formulations with bagacillo.

| Formulation | 1 | 4 | 7 | 11 | 16 | 20 | |
|---|---|---|---|---|---|---|---|
| 1, 2 | 8.3 | 8.6 | 7.7 | 8.5 | 7.6 | 7.6 | |
| 3, 4 | 7.5 | 8.5 | 8.0 | 8.1 | 7.0 | 7.5 | |
| 5, 6 | 8.6 | 8.7 | 7.9 | 8.4 | 7.2 | 7.4 | |
| 7, 8 | 8.2 | 8.5 | 8.3 | 7.2 | 7.1 | 6.9 | |
| 9, 10 | 8.6 | 8.5 | 7.9 | 8.3 | 6.8 | 8.3 | |
| Peat moss | | | | | | | 3.8 |

The soil enhancers and composts determine their utility in intensive agriculture in a large extent by their apparent density. The associate values for an ideal substrate oscillate about of 0.7 g/ml (Pérez, 2006).

The physical effect of application of conditioners or bulk agents on composts is well documented. The reduction of apparent density of substrate leads to a greater capacity of water retention, improves the infiltration and water drainage, and also improves the structure of substrate aggregates.

The results of the effect of formulations of bagacillo appear on the apparent density of composts are submitted in table 7. The results indicate that, for the 5 and 6 formulations, the addition of bagacillo diminished the apparent density of material, in particular for samples of the seventh and sixteenth weeks of said formulations. The sample of the seventh week presented an apparent density of 0.32 g/ml, and constitute the lowest density registered for samples of any formulations. The highest density registered in the 20 weeks of process corresponded only to the control of press mud.

Although for the 1, 2 and 9, 10 formulations, the apparent density was low (between 0.34 and 0.35) in the seventh week, the values are substantially higher than those corresponding to the 5, 6 formulation.

The apparent density of samples of peat moss is between 0.18 and 0.22 g/ml. The density of 0.32 obtained in the 5, 6 formulation in the seventh week is in the apparent density range of an ideal substrate and is the lowest density of all formulations.

TABLE 7
Apparent density of compost samples under
different formulations with bagacillo.

| Formulation | 1 | 4 | 7 | 11 | 16 | 20 | |
|---|---|---|---|---|---|---|---|
| 1, 2 | 0.54 | 0.63 | 0.34 | 0.60 | 0.47 | 0.57 | |
| 3, 4 | 0.61 | 0.67 | 0.65 | 0.70 | 0.67 | 0.80 | |
| 5, 6 | 0.56 | 0.65 | 0.32 | 0.62 | 0.35 | 0.52 | |
| 7, 8 | 0.51 | 0.54 | 0.53 | 0.67 | 0.60 | 0.78 | |
| 9, 10 | 0.51 | 0.56 | 0.35 | 0.60 | 0.35 | 0.54 | |
| Peat Moss | | | | | | | 0.17 |

Another very important parameter for substrates useful in intensive agriculture is the capacity of water retention, which is the maximum amount of water that, after the free drainage, a substrate can retain (Okalebo, 1993).

A high capacity of water retention in a substrate indicates that most of particles has a size from median to small and has a larger area of internal surface, consequently the pores are small, which allows to retain water against the gravity.

Although not all the water a substrate retains is available for its use by the plants, the retention of water available for plants depends that the substrate has a mixture extremely large to fine particles, thereby results in long and small pores, with a bigger proportion of pores of median size. The ratio between texture and capacity of water retention is obvious.

The results of capacity of water retention of the composts under different ratios of starting material are submitted in table 8. The results indicate once again that the 5, 6 formulation in the seventh week demonstrated the greatest capacity of water retention. Said formulation retains 90.45%, with respect to its own dry weight, in comparison with the 123.7% peat moss is able to retain.

Another important parameter for the characterization of substrates for the horticulture use is the distribution of the particle size. The particle size is important because affects the oxygen movement in substrate (through the influence in porosity), and in the access of microbes and enzymes to substrate.

TABLE 8
Percentage of capacity of water retention of compost
samples under different formulations with bagacillo.

| | Weeks | | | | | | |
|---|---|---|---|---|---|---|---|
| Formulation | 1 | 4 | 7 | 11 | 16 | 20 | |
| 1, 2 | 36 | 21 | 42 | 15 | 57 | 63 | |
| 3, 4 | 62 | 55 | 45 | 48 | 54 | 48 | |
| 5, 6 | 39 | 78 | 90 | 48 | 47 | 64 | |
| 7, 8 | 46 | 24 | 41 | 41 | 47 | 55 | |
| 9, 10 | 37 | 32 | 51 | 49 | 77 | 56 | |
| Peat moss | | | | | | | 124 |

Big particles promote the diffusion of oxygen because only their presence means a big pore (Sylvia, 2005). Even so the presence of big particles minimizes the surface of specific area of substrate. That means that most of the substrate is not accessible immediately to microbes and their enzymes.

The mega-particles have a diameter bigger than 1 mm. The big particles are in a range from 0.5 to 1.0 mm, the median are from 0.025 to 0.5 mm, and the fine are less than 0.025 mm (Plaster, 2003).

Benito (2005), emphasizes the importance of the fraction between 0.5 and 1.0 mm, due to the relation with the capacity of water retention of a soil and the water viable for a plant.

The results of the distribution of particle size of the different compost samples under treatment with bagacillo that are submitted in table 9 indicate that for the pilot phase of experimentation, the effect of the treatment with bagacillo generated great differences in the distribution of the particle size of composts.

For the composts of the 5, 6 formulation in the seventh week, we find that the fraction corresponding to particles bigger than 1.98 mm, contains about 24% of the total weight of material. Said fraction favors the existence of macropores, which determine a good drainage of substrate. The fraction of particles between 0.5 and 1.98 mm, constitutes 40% of the total weight of particles, the greatest proportion of the analyzed material. This fraction corresponds to macropores-mesopores, associated to a high capacity of water retention.

The fraction corresponding to the particle size between 0.005 and 0.5 mm constitutes 27%, which corresponds to the fraction of substrate available for microbial activity.

TABLE 9
Percentage of the Particle Size of compost samples
in the different formulations with bagacillo.

| Formulation | 1 | 4 | 7 | 11 | 16 | 20 | |
|---|---|---|---|---|---|---|---|
| Mesh 1.98 mm | | | | | | | |
| 1, 2 | 53.51 | 63.87 | 28.88 | 47.40 | 26.94 | 14.18 | |
| 3, 4 | 32.31 | 36.83 | 37.10 | 35.54 | 22.05 | 24.46 | |
| 5, 6 | 62.41 | 59.44 | 26.20 | 46.72 | 40.89 | 22.48 | |
| 7, 8 | 56.64 | 47.21 | 36.38 | 49.19 | 30.38 | 18.51 | |
| 9, 10 | 60.03 | 46.76 | 28.68 | 39.56 | 30.53 | 21.27 | |
| Peat moss | | | | | | | 12.54 |
| Mesh 0.5 mm | | | | | | | |
| 1, 2 | 32.62 | 32.61 | 21.64 | 48.87 | 35.33 | 40.06 | |
| 3, 4 | 35.02 | 47.44 | 24.46 | 35.12 | 33.19 | 33.49 | |
| 5, 6 | 29.24 | 37.02 | 44.42 | 38.33 | 35.15 | 41.67 | |
| 7, 8 | 31.89 | 39.44 | 45.21 | 37.31 | 38.28 | 49.28 | |
| 9, 10 | 32.75 | 46.00 | 44.99 | 45.77 | 36.60 | 47.43 | |
| Peat moss | | | | | | | 49.20 |
| Mesh 0.025 mm | | | | | | | |
| 1, 2 | 9.87 | 3.28 | 16.96 | 2.95 | 19.63 | 27.46 | |
| 3, 4 | 23.17 | 11.95 | 18.09 | 15.11 | 19.23 | 18.09 | |
| 5, 6 | 5.10 | 3.08 | 17.75 | 9.56 | 13.22 | 23.31 | |
| 7, 8 | 7.64 | 12.04 | 14.60 | 6.99 | 19.47 | 18.10 | |
| 9, 10 | 4.41 | 6.18 | 18.45 | 8.91 | 17.95 | 20.39 | |
| Peat moss | | | | | | | 22.72 |
| Mesh 0.005 mm | | | | | | | |
| 1, 2 | 4.01 | 0.25 | 32.51 | 0.77 | 18.10 | 18.31 | |
| 3, 4 | 9.50 | 3.79 | 20.36 | 14.23 | 25.52 | 23.95 | |
| 5, 6 | 3.26 | 0.45 | 11.63 | 5.39 | 10.74 | 12.53 | |
| 7, 8 | 3.83 | 1.31 | 3.82 | 6.51 | 11.87 | 14.11 | |
| 9, 10 | 2.81 | 1.06 | 7.88 | 5.76 | 14.91 | 10.91 | |
| Peat moss | | | | | | | 15.55 |

The three 5-6, 7-8, 9-10 formulations of example 1, in the seventh and sixteenth week, with the physical properties more similar to peat moss were chosen for the assay of germination, and seeds of grass and tomato, very common cultures of plants needing nitrogen and fast-growing, were used.

The assay of germination focused in the early growth stages of plants, where the deficiencies of nutrients or inhibiting effects are more apparent, and the differences between formulations can be better observed. The numbers of buds satisfactorily emerged from samples of the different selected formulations were counted to obtain the percentage of germinated seeds, and to compare them with the most used organic substrate peat moss. In table 10 it can be observed that 76% of germination for tomato and 72% of germination for grass are attained with the 6, 6 formulation in the seventh week, both percentages are increased to 85% after 5 more days of analysis. In contrast, the percentage of germination from peat moss was very low (47% for grass, and 13% for tomato). In this sense, Wei and collaborators (2005) recognize that a compost with >80% of germination, derived from animal wastes, is considered mature for agricultural use.

The testing plates that were not seeded with seeds of tomato or grass did not register germination of any seed, nor seedling developed in said experimental units. The foregoing indicates that the material is free of viable weed seeds or viable contaminating seeds, as would be expected from a material subjected to composting at high temperature.

In example 2, the substrate produced with the 5, 6 formulation of example 1 was leaded to industrial scale and tested with two controls (2A and 3A) (Table 3).

In this test of example 2a the industrial scale of 100 tons of press mud, the same proportion of 2.3% of weekly additions of bagacillo (1A) was used. Simultaneously, new control biopiles of three tons of starting press mud were established. In the first case 2.3% of bagacillo (control 2A) was added, and 3.3% of bagacillo was added in the second (3A).

TABLE 10
Percentage of germination of compost samples
in the different formulations with bagacillo.

| | |
|---|---|
| Seeds of tomato | Seeds of grass |

| formulations | Week 7 | Week 16 | Week 7 | Week 16 |
|---|---|---|---|---|
| 5, 6 | 76 | 52 | 72 | 18 |
| 7, 8 | 63 | 11 | 39 | 0 |
| 9, 10 | 74 | 9 | 61 | 16 |
| Peat moss | 13 | | 47 | |

TABLE 11
Characteristics of the substrate peat moss and the compost of treatment 5, 6.

| | | | | | | Mesh | Mesh | Mesh | Mesh |
|---|---|---|---|---|---|---|---|---|---|
| | H | TS | pH | BD | % CWR | 1.98 mm | 0.5 mm | 0.025 mm | 0.005 mm |
| 5, 6 | 45 | 55 | 7.9 | 0.32 | 90 | 26.20 | 44.42 | 17.75 | 11.63 |
| Peat moss | 45 | 55 | 3.9 | 0.17 | 247 | 12.54 | 49.20 | 22.72 | 15.55 |

H Humidity
TS Total Solids
BD Bulk Density
% CWR Percentage of Capacity of Water Retention.

Unlike of example 1, and due to the results found therein, the bagacillo was added to the tests of example 2 during six weeks and feedings stopped at the seventh week (after one week of repose), without continuing until the 10 week.

The above described physical parameters of samples of the three biopiles of this example 2 were determined to. Additionally, the porosity was determined and the chemical analysis of the product was carried out. Both peat moss and coconut fiber were utilized as comparison substrates. In table 12, it is possible to see that in the seventh week the substrate of the 1A formulation has 60% of humidity, meanwhile peat moss and coconut fiber have contents of humidity about 46-48%. The 2A and 3A controls have about 40% of the total humidity.

Concerning to pH, the samples of the 1A formulation and their controls, in the first week are found in values near to 5 and as the weeks pass the samples become more alkaline, except that of the 1A formulation which presents values of 4.9 in the last week. Peat moss has a 3.9 pH and coconut fiber has a 6.5 pH (table 12).

As to apparent density, in the sixth and seventh weeks, the substrate of the 1A formulation presents 0.38 g/ml, on average, a value that is in the desired level for an ideal substrate. The 2A and 3A controls have densities between 0.24 and 0.21 g/ml, respectively. The apparent density for peat moss was 0.17 g/ml, and for coconut fiber 0.14 g/ml (table 12).

As to capacity of water retention, the substrate of the 1A formulation has 235% on average in the last week of process, which is the most elevated value with respect to the peat moss values, and is very near to the levels of coconut fiber.

As to particle size, in the last week of composting, the substrate of the 1A formulation has the smallest grams percentage retained in the 1.98 mm mesh, with respect to the 2A and 3A formulations. For the 0.5 mm mesh, the 1A formulation had 43%, a percentage lightly superior to that of the other test windrows. For the smallest fractions (0.025-0.005 mm), the 1A formulation obtained values very near to percentages of same fraction in peat moss and coconut fiber (table 12).

Concerning to the porosity, the 1A formulation has 52%, in comparison with peat moss that presents 56%, that is the 1A formulation and peat moss have a low apparent density and a higher pore space, which is ideal to use in greenhouses and nurseries.

TABLE 12
Physichal - chemical characteristics of materials in composting of the LDHS
and their 3A and 2A controls, collected in the last week of the process.

| | | | | | | Mesh | Mesh | Mesh | Mesh |
|---|---|---|---|---|---|---|---|---|---|
| | H | TS | pH | BD | % CWR | 1.98 mm | 0.5 mm | 0.025 mm | 0.005 mm |
| 1A | 60 | 40 | 4.9 | 0.38 | 235 | 22 | 43 | 24 | 11 |
| 3A | 40 | 60 | 8.2 | 0.21 | 185 | 35 | 41 | 17 | 7 |
| 2A | 41 | 59 | 7.9 | 0.24 | 186 | 33 | 42 | 17 | 8 |
| Coconut | 48 | 52 | 6.5 | 0.14 | 278 | 31 | 36 | 25 | 8 |
| Fiber | | | | | | | | | |
| Peat moss | 46 | 54 | 3.9 | 0.17 | 247 | 13 | 49 | 23 | 16 |

H Humidity
TS Total Solids
BD Bulk Density
% CWR Percentage of Capacity of Water Retention

### Chemical Analysis

The fertility of a substrate is the capacity of same to provide nutrients during the growth of the plant. The substrate can work as a container wherein the nutrients are stored, kept in different forms, some of them more bioavailable than others. The concept of the fertility of a substrate not only includes the amount of nutrients this may store, but also how much are protected from the washings by the effect of rains, how much are available, and how easy are assimilated by the root (Plaster 2003). For the chemical analysis, the sample of the 1A formulation was analyzed in a laboratory of chemical analysis of soils, certificated for this purpose, and wherein the following methodologies were used: Officials Methods of Analysis of AOAC Internacional, Officials Methods of Analysis of APHA (American Public Health Association), Assay carried out by Spectrophotometry of Atomic Absorption/Technique of Flame, Assay carried out by the OLSEN method.

The substrate of the 1A formulation has a composition of: 13.9-23.6% of Organic material, 0.3-0.7% of Total nitrogen, 0.14-0.22% of Potassium, 0.41-0.45% of Calcium, 540-720 ppm of interchangeable Magnesium, 590 ppm of Phosphorus, 240-620 ppm of Bicarbonates, 120-650 ppm of Sulfates, 235-510 ppm of Magnesium, 70-465 ppm of Sodium, 270-310 ppm of Chlorides, 35-65 ppm of zinc.

The substrates with high content in salts are defined as a substrate with 4 or more mohms/cm electrical conductivity. Even so low levels of salinity as 2 mohms/cm can bring about some problems in sensible cultures (of 2-20 mohms/cm). Most of salts are chlorated and sulfates, less than half of cations are sodiums, and a small portion is adsorbed by the colloids of the substrate. The main effect of the salinity is to make more difficult absorb the nutrients of the substrate by the plants. In substrates with very high salinity as coconut fiber, the water is not only attracted by the particles of soil, but is also attracted by the ions in solution, so that less quantity of water is available for the plants. The substrate of the 1A formulation has a conductivity range of 2.7-3 mohms/cm, although it has low levels of salinity, may present problems in some very sensible cultures.

### Microbiologic Analysis

Samples of LDHS were analyzed by the use of culture resources that promote the growth of pathogenic fungi, such as Potato-Dextrose-Agar (PDA), Sabouraud, Malt Extract-Agar (MEA), and VPN3. The pathogenic fungi typically associated with agricultural soils and greenhouses, of types such as Verticillium, Pythium, Rhizoctonia, Fusarium, Phytophthora, Sclerotium or Colletotrichum, among others, were absent from the referred resources, at incubation temperatures between 25 and 30° C. The fungi found in said resources grew in a great proportion at 45° C., and belong to types typically associated to high temperature composts, such as Penicillium, Phanaerochaete, Rhizopus and Thermomucor, among others, none of which is a known plant pathogen, nor reason for radicular or systemic pathogenesis (Rouxel y Francis, 2000; Singleton et al, 1992).

### General Description of the Low-Density Humectant Substrate (LDHS)

It is a product generated from an improved process of composting from agro-industrial residues or harvests, more preferably lignocellulosic residues, even more preferably fibrous lignocellulosic residues, more preferably residues of sugar mills, corn, agave, straws of gramineous plants and grain husks such as rice and barley. Still more preferably, of bagacillo of cane, bagasse, press mud or mud of filtration. It is generated in semi-static biopiles, called fed batch, with additions in different stages of lignocellulosic material. The time of treatment is very short (almost two months) as compared to the processing time of a mature compost, which typically is from 12 to 24 weeks, and during the composting, temperatures between 60 and 85° C. are reached, enabling the elimination of seeds of weeds, as well as of fungi and pathogenic bacteria.

As starting raw material, the LDHS contains press mud. For additions or feedings of lignocellulosic fiber, bagacillo is mainly used, although any residue with high content of lignocellulosic fiber can be used. The alternative materials can be: full cane bagasse (crust and marrow), bagasse of agave, straws of corn and of other gramineous plants, and grain husks such as rice and barley.

The process of composting to produce the LDHS is a very flexible process that allows us to modify different steps of the treatment in order to obtain variations of the LDHS with different characteristics and qualities, depending of the intented use therefor. The LDHS in its different forms can be used as covering grounds, enhancer agents and for the bulk of soils, humectant agents, biofertilizers, and integral substrates for horticulture and forestal production in greenhouses and nurseries.

The samples of LDHS derived from production of 100 tons have been used as a sole substrate for intensive production of tomato within a greenhouse. The results of germination and initial growth of the plants, indicate that the substrate is superior to peat moss as a sole substrate.

In the same way, when the LDHS was applied in a furrow of potato production, in an agricultural field, the weight and quality of the product were improved, presumably by its humectant function and of soil improvement.

### BIBLIOGRAPHICAL REFERENCES

Baca M. T., Esteban E., Almendros G. and Sanches-Raya A. J., Changes in the gas phase of compost during solid state fermentation of sugarcane Bagase. (1993) Bioresource Technology 44 5-8.
Benito M., Masaguer A., De Antonio R. and Moliner A., Use of pruning waste compost as a component in soilless growing media. (2005) Bioresource Technology 96 597-603.
Bertran E., Sort X., Soliva M. and Trillas I., Composting winwry waste: sludge and grpa stalks. (2004) Bioresource Technology 95 203-208.
Bodin D. and Thorup-Kristensen K., Delayed nutrient application affects mineralization rate during composting of plant residues. (2005) Bioresource Technology 96 1093-3101.
Ekinci K., Keener H. M. and Elwell D. L., Composting short paper fiber with broiler litter and additives. (2000) Compost science and Utilization, Vol. 8, No. 2, 160-172.
Garcia-Gomez A., Bernal M. P. and Roig A., Growth of ornamental plants in two composts prepared from agroindustrial wastes. (2002) Bioresource Technology 83 81-87.
Haug T. H., The principal handbook of compost engineering. Ed. Lewis Publishers, BocaRaton, Fla., 1993.
Hernandez A. L., Gascó M. A., Gascó M. J. and Guerrero F., Reuse of waste materials as growing media for ornamental plants. (2005) Bioresource Technology 96 125-131.
Kohnke H. and Franzmeier D. P., Soil science simplified. Editorial Waveland, 4° ed, 1995, USA; pp. 1-53.
Meunchang S., Panichsakapatana S., Weaver R. W., Co-composting of filter cake and bagasse by-products from a sugar mill. (2005) Bioresource Technology 96: 437-442
Okalebo J. R. and Gathua W. K., Laboratory methods of soil and plant analysis: A working manual. Editorial KARY SSSEA TSBF UNESCO-ROSTA, 1993, Nairobi, Kenya; pp. 1-87.
Paredes C., Cegarra J., Bernal M. P. and Roig A., Influence of olive mill wastewater in composting and impact of the compost on a Swiss chard crop and soil properties. (2005) Enviroment International 31 305-312
Perez-Murcia D. M., Moral R., Caselles-Moreno J., Perez-Espinosa A. and Paredes C., Use of composted swage sludge in growth media for broccoli. (2006) Bioresource Technology 97 123-130.
Plaster J. E., Soil science and management. Editorial Delmar Learning, 4° ed, 2003, USA; pp. 246-257.
Prince W., Sivakumar S., Ravi V. and Subburam V., The effects of coirpith compost on the growth and quality of leaves of the mulberry plant Morus alba L. (2000) Bioresource Technology 72 95-97.
Ram D., Ram M. and Singh R., Optimization of water and nitrogen application to menthol mint (Mentha arvensis L.) through sugarcane trash mulch in a sandy loam of semi-arid subtropical climate. (2006) Bioresource Technology 97 886-893.
Rechcigl E. J., Soil amendments and environmental quality. Editorial Lewis Publisher, 1995, USA; pp. 249-327.
Rechcigl E. J., Soil amendments impacts on biotic systems. Editorial Lewis Publisher, 1995, USA; pp. 2-30.
Rosas-Morales M., Mejoramiento del composteo de los derivados de la caña de azúcar: cachaza y bagacillo. Tesis de Maestría. Programa de Ciencias Ambientales. Instituto de Ciencias de la Benemérita Universidad Autónoma de Puebla. México, 2003, pp. 1-76.
Rouxel I. and Francis I. I., Detection and Isolation of Soil Fungi, 2000, Science Publishers Inc. USA.
Simone L. J. and Taylor R. B., Effects of pulp mill solids and three composts on early growth of tomatoes. (2003) Bioresource Technology 89 297-305.
Sánchez R., Palma J., Obrador J. and Lopez U., Efecto de los rastrojos sobre las propiedades fisicas y químicas de un suelo vertisol y rendimiento de caña de azúcar (Saccharum officinarum L.) en Tabasco, México. (July 2003) Interciencia Vol. 28, No. 7, 404-407
Singleton L. A., Mihail J. D., Rush C. M., Methods for research on soilborne phytopathogenic fungi (1992) American Phytopathological Society, USA.
Soumaré M., Tack F. M. G. and Verloo M. G., Effects of a municipal solid waste compost and mineral fertilization on plant growth in two tropical agricultural soils of Mali. (2003) Bioresource Technology 86 15-20.
Stabnikova O., Goh W. K., Ding H., Tay J. and Wang J., The use of swage sludge and horticultural waste to develop artificial soil of plant cultivation in Singapore. (2005) Bioresource Technology 96 1073-1080.
Stoffella P. and Graetz D., Utilization of sugarcane compost as a soil amendment in a tomato production system. (2000) Compost science and Utilization, Vol. 8, No. 3, 210-214.
Sylivia M. D., Hartel G. P., Furhmann J. J., and Zuberer A. D., Principles and applications of soil microbiology. Editorial Pearson Education Inc., 2° ed, 2005, USA; pp. 587-605
Valdés M. and Medina J. N., Ecología Microbiana del suelo Compendio práctico. Editorial IPN, 1^{a} ed. D. F., México, 2005.
Wallace A. and Richard E. T., Handbook of Soil Conditioners: substances that enhance the physical properties of soil. Editorial Marcel Dekker, 1998, New York, USA; pp. 43-96.
Warman P. R. and Termeer W. C., Composting and evaluation of racetrack manure, grass clippings and sewage sludge. (1996) Bioresource Technology 55 95-101.
Wei Y. S.; Fan Y. B., Wang M. J., Wang J. S. Composting and compost application in China. 2000. Resources, Conservation and Recycling 30, 277-300.
Wilson S. and Stoffella P., Evaluation of compost as an amendment to commercial mixes used for container-grown golden shrimp plant production. (January-March 2001) HortTechnology 11(1), 31-34
Patent No. 20050284202, of Rampton Lea et al.
Patent No. 20050274074 of Stamp John Wesley.
Patent No. WO1994022790 of Bandurski, William E.
Patent No. WO2003002638 of ONG, Tet, Siong.
Infozafra 2005/2006 www.caneros.org.mx

1. A process of composting by semi-static biopiles in order to obtain a low-density humectant substrate which includes the step to provide the starting materials which consist of fibrous lignocellulosic materials; form biopiles or windrows; mix the lignocellulosic starting materials, homogenize uniformly the starting materials; leave to repose the starting materials; add fresh lignocelluloses; mix and leave to repose again; stabilize the composted material by drying; the process is characterized because the steps to add fresh lignocelluloses, mix and leave to repose the starting materials are periodically repeated 5 times at least.

2. The process of composting by semi-static biopiles in order to obtain a low-density humectant substrate according to claimed in claim 1, characterized because the starting materials are selected from residues of sugar mills, residues of agave, residues of corn, straws of gramineous plants and grain husks of wheat, corn, rice and barley.

3. The process of composting by semi-static biopiles in order to obtain a low-density humectant substrate according to claimed in claim 2, characterized because the starting materials are selected from bagacillo of cane, bagasse, press mud, or mud of filtration.

4. The process of composting by semi-static biopiles in order to obtain a low-density humectant substrate according to claimed in claim 1, characterized because the step to provide the starting materials includes a proportion of press or muds of filtration of bagasses or bagacillos of cane between 10:1 and 83:1.

5. The process of composting by semi-static biopiles in order to obtain a low-density humectant substrate according to claimed in claim 1, characterized because the step to provide the starting materials includes a proportion of press or muds of filtration of bagasse of cane of between 30:1 and 43:1.

6. The process of composting by semi-static biopiles in order to obtain a low-density humectant substrate according to claimed in claim 1, characterized because the step to provide the starting materials includes a proportion of press or muds of filtration:bagasse of cane of 43:1.

7. The process of composting by semi-static biopiles in order to obtain a low-density humectant substrate according to claimed in claim 1, characterized because the formation of biopiles is carried out on an absorbent litter of bagasses or straws with a thickness about 50 to 60 cm.

8. The process of composting by semi-static biopiles in order to obtain a low-density humectant substrate according to claimed in claim 1, characterized because the step of mixing is carried out with a mechanical composter.

9. The process of composting by semi-static biopiles in order to obtain a low-density humectant substrate according to claimed in claim 1, characterized because the step to leave to repose the starting material includes a one week time.

10. The process of composting by semi-static biopiles in order to obtain a low-density humectant substrate according to claimed in claim 1, characterized because the addition of fresh lignocelluloses is, in each dose, between 0.75 and 5% weight, with respect to weight of press mud in the starting mixture.

11. The process of composting by semi-static biopiles in order to obtain a low-density humectant substrate according to claimed in claim 1, characterized because in the process, between the starting mixture and subsequent additions, the total added lignocellulose has a total weight equivalent between 6 and 35% of weight of the initial fresh press mud.

12. The process of composting by semi-static biopiles in order to obtain a low-density humectant substrate according to claimed in claim 1, characterized because the additional reposes are of one week too.

13. The process of composting by semi-static biopiles in order to obtain a low-density humectant substrate according to claimed in claim 1, characterized because the drying step to stabilize the composted material is carried out at ambient temperature until the composted material has an humidity content between 20 and 35%.

14. The process of composting by semi-static biopiles for obtaining a low-density humectant substrate according to claimed in claim 1, characterized because during the first repetition of steps to add fresh lignocelluloses, mix and leave to repose the starting materials, temperatures between 50° C. and 60° C. are reached and during subsequent repetitions of steps to add fresh lignocelluloses, mix and leave to repose the starting materials, temperatures between 65° C. and 85° C. are reached.

15. The process of composting by semi-static biopiles in order to obtain a low-density humectant substrate according to claimed in claims 1 to 14, characterized because said process is carried at the open air.

16. The process of composting by semi-static biopiles in order to obtain a low-density humectant substrate according to claimed in claims 1 to 14, characterized because said process is carried out in a 7 to 8 weeks period.

17. The process of composting by semi-static biopiles in order to obtain a low-density humectant substrate according to claimed in claims 1 to 16, wherein said substrate has a range of total humidity from 40 to 60% and a range of total solids from 60 to 40%, and it is equilibrated in <30% under sun drying.

18. The process of composting by semi-static biopiles in order to obtain a low-density humectant substrate according to claimed in claims 1 to 16, wherein said substrate has a pH range between 5.0 and 8.0.

19. The process of composting by semi-static biopiles in order to obtain a low-density humectant substrate according to claimed in claims 1 to 16, wherein said substrate has a density range between 0.21 and 0.48 g/ml.

20. The process of composting by semi-static biopiles in order to obtain a low-density humectant substrate according to claimed in claims 1 to 16, wherein said substrate has an ability of water retention range between 80 to 130% with respect to its weight.

21. The process of composting by semi-static biopiles in order to obtain a low-density humectant substrate according to claimed in claims 1 to 16, wherein said substrate has a granulometry range between 21 and 30% of particles bigger than 1.98 mm, between 30 and 45% of particles with a size between 0.5 and 1.98 mm, and between 10 and 30% of particles with a size between 0.005 and 0.5 mm.

22. The process of composting by semi-static biopiles in order to obtain a low-density humectant substrate according to claimed in claims 1 to 16, wherein due to the distribution of particle size, said substrate has a very low density, high humidity retention, and great availability of water for plant.

23. The process of composting by semi-static biopiles in order to obtain a low-density humectant substrate according to claimed in claims 1 to 16, wherein said substrate has a porosity between 50 and 60%.

24. The process of composting by semi-static biopiles in order to obtain a low-density humectant substrate according to claimed in claims 1 to 16, wherein said substrate is free of fungi pathogenic to plants and seeds or seedlings of weeds due to the treatment at high temperature.

25. The process of composting by semi-static biopiles in order to obtain a low-density humectant substrate according to claimed in claims 1 to 16, wherein said substrate is useful as sole substrate or as main substrate for the production of plants in systems of intensive agriculture in a greenhouse and a nursery.

26. The process of composting by semi-static biopiles in order to obtain a low-density humectant substrate according to claimed in claims 1 to 16, wherein said substrate is useful for its application as humectant and enhancer agent in agricultural soils.
